(19) United States
(12) Patent Application Publication
(10) Pub. No.: US 2026/0335281 A1
(43) Pub. Date: Oct. 29, 2026

(54) PHARMACEUTICAL COMPOSITION COMPRISING CENTANAFADINE

(71) Applicant: OTSUKA PHARMACEUTICAL CO., LTD., Tokyo (JP)

(72) Inventor: Masahiro Hasegawa, Osaka (JP)

(21) Appl. No.: 19/105,706

(22) PCT Filed: Aug. 23, 2023
(86) PCT No.: PCT/JP2023/030255

§ 371 Date: Feb. 21, 2025

(30) Foreign Application Priority Data
Aug. 24, 2022 (JP) 2022-133266

(51) Int. Cl. A61K 31/403; A61K 9/50

(52) U.S. Cl. CPC A61K 31/403; A61K 9/501; A61K 9/5015; A61K 9/5026; A61K 9/5047; A61K 9/5073

(57) ABSTRACT

The present invention relates to a pharmaceutical composition comprising (1R,5S)-1-(naphthalen-2-yl)-3-azabicyclo[3.1.0]hexane (centanafadine) or a pharmaceutically acceptable salt thereof as an active ingredient, and a formulation comprising the pharmaceutical composition, which can be administered once daily.

### TECHNICAL FIELD

The present invention relates to a pharmaceutical composition comprising (1R,5S)-1-(naphthalen-2-yl)-3-azabicyclo[3.1.0]hexane (centanafadine) or a pharmaceutically acceptable salt thereof as an active ingredient, and an oral solid dosage formulation comprising the pharmaceutical composition, which can be administered once daily.

### BACKGROUND ART

(1R,5S)-1-(Naphthalen-2-yl)-3-azabicyclo[3.1.0]hexane is also known as (+)-1-(naphthalen-2-yl)-3-azabicyclo[3.1.0]hexane, which can inhibit the reuptake of three biogenic amines: norepinephrine, serotonin, and dopamine (PL 1).

(1R,5S)-1-(Naphthalen-2-yl)-3-azabicyclo[3.1.0]hexane is the most effective against norepinephrine (NE) reuptake, but the effectiveness against dopamine (DA) reuptake is 6-fold less and the effectiveness against serotonin (5-HT) reuptake is 14-fold less. Thus, (1R,5S)-1-(naphthalen-2-yl)-3-azabicyclo[3.1.0]hexane is a compound useful as an unbalanced triple reuptake inhibitor (TRI). Based on these actions, the present compound is useful as a therapeutic agent for CNS disorders such as depression, anxiety, and attention deficit hyperactivity disorder (ADHD).

Recently, once-daily oral dosing is recommended for drug use in the treatment of CNS disorders such as depression, anxiety, and attention deficit hyperactivity disorder (ADHD). Pharmaceutical compositions suitable for once-daily oral administration have been developed, for example, a pharmaceutical formulation comprising multiple drug-containing granules has been developed, which is prepared with delayed-release (enteric) particle, sustained release particle, and/or immediate release particle.

### CITATION LIST

### Patent Literature

[PL 1] JP 5184354 B

### SUMMARY OF INVENTION

### Technical Problem

One of the problems to be solved in the present invention is to provide a pharmaceutical composition comprising (1R,5S)-1-(naphthalen-2-yl)-3-azabicyclo[3.1.0]hexane (centanafadine, hereinafter, also referred to as “Compound (I)”) or a salt thereof, which is suitable for once-daily oral administration.

### Solution to Problem

The present inventors have extensively studied to solve the above problem and then have found that a pharmaceutical composition suitable for maintaining sustained absorption in the lower gastrointestinal tract can be prepared by coating core particles comprising Compound (I) or a salt thereof with at least two types of polymers.

The present invention includes the following embodiments.

### (Item 1)

A pharmaceutical composition comprising a core particle with a coating layer, wherein the core particle comprises (1R,5S)-1-(naphthalen-2-yl)-3-azabicyclo[3.1.0]hexane or a salt thereof as an active ingredient, and wherein the coating layer comprises at least two types of polymers.

### (Item 2)

The pharmaceutical composition of Item 1, wherein the two types of polymers are an enteric polymer and a sustained-release polymer.

### (Item 3)

The pharmaceutical composition of Item 2, wherein the enteric polymer is methacrylic acid copolymer LD.

### (Item 4)

The pharmaceutical composition of Item 2 or 3, wherein the sustained-release polymer comprises one or more polymer(s) selected from alkylcellulose, acrylic acid polymer, acrylic acid copolymer, vinyl acetate polymer and cellulose ether.

### (Item 5)

The pharmaceutical composition of Item 4, wherein the sustained-release polymer comprises one or more polymer(s) selected from alkylcellulose and acrylic acid copolymer.

### (Item 6)

The pharmaceutical composition of Item 5, wherein the alkylcellulose is ethylcellulose.

### (Item 7)

The pharmaceutical composition of Item 5, wherein the acrylic acid copolymer is ethyl acrylate-methyl methacrylate copolymer.

### (Item 8)

The pharmaceutical composition of Item 2, wherein the enteric polymer is methacrylic acid copolymer LD, and wherein the sustained-release polymer is ethylcellulose.

### (Item 9)

The pharmaceutical composition of Item 2, wherein the enteric polymer is methacrylic acid copolymer LD, and wherein the sustained-release polymer is ethyl acrylate-methyl methacrylate copolymer.

### (Item 10)

The pharmaceutical composition of any one of Items 1 to 9, wherein the coating layer is composed of multi-layer.

### (Item 11)

The pharmaceutical composition of Item 10, wherein the coating layer is composed of two coating layers: a coating layer comprising enteric polymer and a coating layer comprising sustained-release polymer, wherein the coating layer of enteric polymer covers the coating layer of sustained-release polymer.

### (Item 12)

The pharmaceutical composition of Item 11, wherein the amount of sustained-release polymer in case of multi coating layer is about 2 wt % to about 40 wt %, or about 5 wt % to about 15 wt %, or about 8 wt % to about 12 wt %, or about 10 wt % per the total weight of the core particle or the seal-coated core particle.

### (Item 13)

The pharmaceutical composition of Item 11 or 12, wherein the amount of enteric polymer in case of multi coating layer is about 10 wt % to about 50 wt %, or about 10 wt % to about 40 wt %, or about 15 wt % to about 30 wt %, or about 15 wt % to about 25 wt % per the total weight of the core particle or the seal-coated core particle.

### (Item 14)

The pharmaceutical composition of any one of Items 1 to 9, wherein the coating layer is composed of a single-layer.

### (Item 15)

The pharmaceutical composition of Item 14, wherein the single coating layer comprises sustained-release polymer and enteric polymer.

### (Item 16)

The pharmaceutical composition of Item 15, wherein the amount of sustained-release polymer in case of single coating layer is about 2 wt % to about 40 wt %, or about 5 wt % to about 15 wt %, or about 6 wt % to about 13 wt % per the total weight of the core particle or the seal-coated core particle.

### (Item 17)

The pharmaceutical composition of Item 15 or 16, wherein the amount of enteric polymer in case of single coating layer is about 10 wt % to about 50 wt %, or about 15 wt % to about 40 wt %, or about 20 wt % to about 40 wt % per the total weight of the core particle or the seal-coated core particle.

### (Item 18)

The pharmaceutical composition of any one of Items 15 to 17, wherein the weight ratio of the sustained-release polymer to the enteric polymer in the coating layer is 1:1 to 1:5.

### (Item 19)

The pharmaceutical composition of Item 18, wherein the weight ratio of the sustained-release polymer to the enteric polymer in the coating layer is 1:3.

### (Item 20)

The pharmaceutical composition of any one of Items 1 to 19, wherein the core particle is seal-coated.

### (Item 21)

The pharmaceutical composition of any one of Items 1 to 20, wherein the core particle with the coating layer is a delayed-release (enteric) particle.

### (Item 22)

The pharmaceutical composition of any one of Items 1 to 21, wherein the active ingredient is (1R,5S)-1-(naphthalen-2-yl)-3-azabicyclo[3.1.0]hexane hydrochloride.

### (Item 23)

The pharmaceutical composition of any one of Items 1 to 22, which meets the followings:
a) less than 10% of the active ingredient is released at 2 hours, and
b) 80% or more of the active ingredient is released at 4 to 12 hours, when the pharmaceutical composition is tested according to Apparatus for Basket Method in the Japanese Pharmacopoeia dissolution test at 37° C.±0.5° C. at 100 rpm, first in 900 mL of 0.1 N HCl solution for 2 hours, and then in 900 mL of pH 7.4 buffer solution for 10 hours.

### (Item 24)

The pharmaceutical composition of any one of Items 1 to 22, which meets the followings:
a) less than 10% of the active ingredient is released at 2 hours,
b) 30% to 70% of the active ingredient is released at 4 hours, and
c) 80% or more of the active ingredient is released at 12 hours, when the pharmaceutical composition is tested according to Apparatus for Basket Method in the Japanese Pharmacopoeia dissolution test at 37° C.±0.5° C. at 100 rpm, first in 900 mL of 0.1 N HCl solution for 2 hours, and then in 900 mL of pH 7.4 buffer solution for 10 hours.

### (Item 25)

The pharmaceutical composition of any one of Items 1 to 24, which is for use in preventing and/or treating central nervous system (CNS) disorder.

### (Item 26)

The pharmaceutical composition of Item 25, wherein the central nervous system (CNS) disorder is at least one CNS disease selected from the group consisting of schizophrenia; treatment-resistant, intractable, or chronic schizophrenia; emotional disorder; psychotic disorder; mood disorder; bipolar disorder; mania; depression; endogenous depression; major depression; melancholia and treatment-resistant depression; dysthymic disorder; cyclothymic disorder; anxiety disorder; somatoform disorder; factitious disorder; dissociative disorder; sexual disorder; eating disorder; sleep disorder; adjustment disorder; substance-related disorder; anhedonia; delirium; cognitive impairment; Alzheimer's disease; Parkinson's disease; cognitive impairment associated with other neurodegenerative diseases; BPSD associated with dementia; cognitive impairment in schizophrenia; cognitive impairment due to treatment-resistant, intractable, or chronic schizophrenia; vomiting; motion sickness; obesity; migraine; pain; mental retardation; autism; Tourette's disorder; tic disorder; attention deficit hyperactivity disorder; conduct disorder; borderline personality disorder; nicotine-related disorder; bulimia; and Down syndrome.

### (Item 27)

Use of the pharmaceutical composition of any one of Items 1 to 24, in the manufacture of a medicament for preventing and/or treating central nervous system (CNS) disorder.

### (Item 28)

An oral solid dosage formulation comprising the pharmaceutical composition of any one of Items 1 to 26.

### (Item 29)

The oral solid dosage formulation of Item 28, which is granules or capsules.

### (Item 30)

The oral solid dosage formulation of Item 28 or 29, further comprising a sustained release particle comprising (1R,5S)-1-(naphthalen-2-yl)-3-azabicyclo[3.1.0]hexane or a salt thereof and/or an immediate release particle comprising (1R,5S)-1-(naphthalen-2-yl)-3-azabicyclo[3.1.0]hexane or a salt thereof.

### Effect of Invention

The pharmaceutical composition comprising a core particle with a coating layer of the present invention is designed as a delayed-release (enteric) particle suitable for maintaining sustained absorption of Compound (I) or a salt thereof in the lower gastrointestinal tract. An oral solid dosage formulation comprising the pharmaceutical composition of the present invention can be used in the form of capsules or granules.

In addition, the pharmaceutical composition of the present invention can be used in combination with another sustained release particle comprising Compound (I) or a salt thereof and/or an immediate release particle comprising Compound (I) or a salt thereof. Furthermore, the pharmaceutical composition of the present invention in combination with the other sustained release particle and/or the immediate release particle can be used as capsules or granules.

### BRIEF DESCRIPTION OF DRAWINGS

FIG. 1 shows the result of dissolution test in Example 5, in which the particles prepared in Example 4-1 and Example 4-2 were tested.

FIG. 2 shows the result of dissolution test in Example 8, in which the particles prepared in Example 7-1 and Example 7-2 were tested.

FIG. 3 shows the result of dissolution test in Example 10, in which the particles prepared in Example 9-1 and Example 9-2 were tested.

FIG. 4 shows the result of dissolution test in Comparative example 2, in which the particles prepared in Comparative example 1-1 to Comparative example 1-3.

### DESCRIPTION OF EMBODIMENTS

The terms and the like used herein are explained below.

The pharmaceutical composition comprising a core particle with a coating layer of the present invention is designed as a delayed-release (enteric) particle suitable for maintaining sustained absorption of Compound (I) or a salt thereof in the lower gastrointestinal tract.

The pharmaceutical composition encompassed by the present invention comprises (1R,5S)-1-(naphthalen-2-yl)-3-azabicyclo[3.1.0]hexane [Compound (I)] or a salt thereof as an active ingredient, which is shown as Compound (I) below. Compound (I) may be in any form, for example, free form or pharmaceutically acceptable salt form. The salt of Compound (I) should not be limited as long as the salt is a pharmaceutically acceptable one, which includes, preferably for example, an acid addition salt thereof, more preferably for example, hydrochloride of Compound (I).

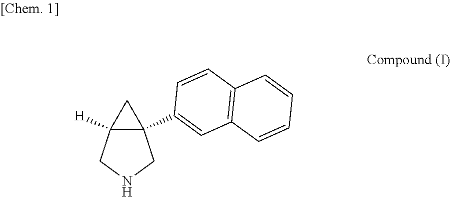 [SMILES: c1ccc2cc([C@]34CNC[C@H]3C4)ccc2c1]

As used herein, Compound (I) or a salt thereof is understood to encompass the compound in crystalline form or in amorphous form, which includes a polymorph, a solvate including hydrate, a non-solvate, a conformational polymorph, and a mixture thereof. In the present invention, the “crystalline form” and the “polymorph” are sometimes used interchangeably, and the present invention is intended to encompass all compounds of Compound (I) or a salt thereof in free form or in pharmaceutically acceptable salt form, which includes a polymorph, a solvate including hydrate, a non-solvate, a conformational polymorph, and a mixture thereof, unless otherwise indicated.

In addition, Compound (I) or a salt thereof encompasses an isomer such as a geometric isomer, stereoisomer, and optical isomer thereof.

The pharmaceutical composition comprising a core particle with a coating layer of the present invention is designed as a delayed-release (enteric) particle, and the delayed-release (enteric) particle has an inner core and an outer layer, wherein the inner core is “a core particle comprising Compound (I) or a salt thereof”, and the outer layer is “a coating layer comprising at least two types of polymers”. The core particle comprising Compound (I) or a salt thereof may be seal-coated.

The core particle comprises Compound (I) or a salt thereof as an active ingredient and additives. Preferably, it comprises hydrochloride of Compound (I) as an active ingredient and additives.

The amount of Compound (I) or a salt thereof in the core particle is about 5 wt % to about 95 wt % per the total weight of the core particle, preferably about 20 wt % to about 90 wt %, more preferably about 30 wt % to about 90 wt %, even more preferably about 40 wt % to about 85 wt %, the most preferably about 50 wt % to about 80 wt %.

The additives in the core particle may include one or more materials selected from excipient, binder, glidant, surfactant, lubricant, disintegrant, and plasticizer. The preferred additives are excipient and/or binder.

The excipient includes, for example, lactose, sucrose, glucose, corn starch, microcrystalline cellulose, mannitol, sorbitol, calcium hydrogen phosphate, aluminum silicate, amorphous silica, and sodium chloride, but not limited thereto. These ingredients may be used alone or in combination of two or more.

The amount of excipient in the core particle is about 5 wt % to about 95 wt % per the total weight of the core particle, preferably about 10 wt % to about 90 wt %, more preferably about 15 wt % to about 90 wt %, even more preferably about 20 wt % to about 90 wt %, the most preferably about 20 wt % to about 50 wt %.

The binder includes cellulose ether, methylcellulose, ethylcellulose, hydroxyethyl-cellulose, propylcellulose, hydroxypropylcellulose, low substituted hydroxypropylcellulose, hypromellose (HPMC), carboxymethylcellulose, starch, pregelatinized starch, acacia, tragacanth, gelatin, polyvinylpyrrolidone (povidone, PVP), polyvinyl alcohol-polyethylene glycol graft copolymer, crosslinked polyvinylpyrrolidone, sodium alginate, and microcrystalline cellulose, but not limited thereto. These ingredients may be used alone or in combination of two or more.

The glidant includes polyethylene glycols of various molecular weights, magnesium stearate, calcium stearate, calcium silicate, silicon dioxide, magnesium carbonate, magnesium lauryl sulfate, aluminum stearate, stearic acid, palmitic acid, cetanol, stearyl alcohol, and talc, but not limited thereto. These ingredients may be used alone or in combination of two or more.

The surfactant includes anionic surfactants such as sodium lauryl sulfate, sodium deoxycholate, dioctyl sodium sulfosuccinate, and sodium stearyl fumarate; nonionic surfactants such as polyoxyethylene ethers and polysorbate 80; cationic surfactants such as quaternary ammonium compounds, but not limited thereto. These ingredients may be used alone or in combination of two or more.

The lubricant includes stearic acid, magnesium stearate, calcium stearate, aluminum stearate, stearyl alcohol fumarate, and siliconized talc, but not limited thereto. These ingredients may be used alone or in combination of two or more.

The disintegrant includes starch, crosslinked carboxymethylcellulose sodium, croscarmellose sodium, croscarmellose calcium, crosslinked polyvinylpyrrolidone, sodium starch glycolate, low substituted hydroxypropylcellulose, and hydroxypropyl starch, but not limited thereto. These ingredients may be used alone or in combination of two or more.

The plasticizer includes butyl citrate, triethyl citrate, diethyl phthalate, dibutyl sebacate, PEG (e.g. PEG 6000), acetyltriethyl citrate, and triacetin, but not limited thereto. These ingredients may be used alone or in combination of two or more.

The core particle is a homogeneous dry composition formed, for example, by compressing a mixture of Compound (I) and additives. The core particle may be formed, for example, from a particle of milled Compound (I) or salt thereof and additives. The core particle can be prepared using known techniques, including but not limited to, granulation, spray-drying, sieving, lyophilizing, pulverizing, milling, and micronizing. The preparation of the core particle can be carried out using the following devices: for example, stirring granulator, extrusion granulator, fluid bed granulator, spherical granulator, and roller compactor.

The core particle may be seal-coated. The purpose of seal coating is to smooth the surface of the core particle, which is carried out by coating the surface of the core particle, for example, with a coating ingredient comprising water-soluble polymer.

The seal-coating method is not particularly limited, but it can be carried out by coating a core particle containing Compound (I) or a salt thereof with a seal coating liquid in a coating machine such as a fluid bed granulator.

As the coating ingredient, a known coating agent may be used, which includes, for example, hydrophilic polymers, more specifically, hydrophilic cellulose ethers, preferably hypromellose (HPMC), hydroxypropylcellulose, methylcellulose, polyvinylpyrrolidone, polyvinyl alcohol, polyvinyl alcohol-polyethylene glycol graft copolymer (Kollicoat IR), and polyethylene glycol, but not limited thereto. These ingredients may be used alone or in combination of two or more, and the coating ingredient may further comprise a plasticizer or an anti-tacking agent. Preferably, hypromellose (HPMC) may be used.

The amount of seal coating ingredient for the core particle is about 1 wt % to about 10 wt % per the total weight of the core particle, preferably about 2 wt % to about 8 wt %, more preferably about 2 wt % to about 6 wt %.

The delayed-release (enteric) particle is prepared by coating the above-mentioned core particle or the above-described seal-coated core particle, with a coating layer comprising at least two types of polymers, wherein the coating layer is composed of a single layer or multi layers (more than 2 layers).

The coating layer comprising two types of polymers may comprise an enteric polymer and a sustained-release polymer, wherein said coating layer may be single-layer or multi-layer.

The single-layer-coated particle can be prepared by coating a core particle or a seal-coated core particle with a coating liquid comprising a sustained-release polymer and an enteric polymer, followed by drying the same.

The multi-layer-coated particle includes, for example, two-layer-coated particle, three-layer-coated particle, and four-layer-coated particle, and preferably two-layer-coated particle, but not limited thereto. The two-layer-coated particle can be prepared by coating a core particle or a seal-coated core particle with a coating liquid comprising a sustained-release polymer, followed by drying the same to give a sustained-release-coated particle, and then by further coating the sustained-release-coated particle with a coating liquid comprising an enteric polymer, followed by drying the same.

For both single-layer-coated particle and multi-layer-coated particle, the coating with a coating liquid can be done according to conventional methods.

Such conventional coating methods are not particularly limited, but which can be done by coating a core particle or a seal-coated core particle by using a coating machine such as a fluid bed granulator.

The sustained-release polymer includes alkylcellulose, acrylic acid polymer, methacrylic acid polymer, acrylic acid copolymer, methacrylic acid copolymer, vinyl acetate polymer, cellulose ether, hydroxyalkylcellulose, and carboxyalkylcellulose. Preferably, it includes ethylcellulose, ethyl acrylate-methyl methacrylate-trimethylammonioethyl chloride copolymer, hydroxypropyl methylcellulose (hypromellose (HPMC)), ethyl acrylate-methyl methacrylate copolymer, ammonioalkyl methacrylate copolymer, and polyvinylacetic acid-polyvinylpyrrolidone copolymer. More preferably, it includes ethylcellulose, and ethyl acrylate-methyl methacrylate copolymer. The ethylcellulose includes, for example, Aquacoat™ ECD-30. The ethyl acrylate-methyl methacrylate copolymer is a copolymer of ethyl acrylate and methyl methacrylate, wherein the content ratio of ethyl acrylate and methyl methacrylate is about 2:1. The ethyl acrylate-methyl methacrylate copolymer includes, for example, Eudragit™ NE30D and Eudragit™ NM30D.

The enteric polymer herein is a polymer which is dissolved at pH 4.5 or higher, which includes, for example, methacrylic acid copolymer LD and methacrylic acid-ethyl acrylate copolymer. The methacrylic acid-ethyl acrylate copolymer is a copolymer of methacrylic acid and ethyl acrylate, wherein the content ratio of methacrylic acid and ethyl acrylate is about 1:1. The methacrylic acid-ethyl acrylate copolymer includes, for example, Eudragit™ L30D-55.

The amount of sustained-release polymer in both cases of multi coating layer and single coating layer is about 2 wt % to about 40 wt % per the total weight of the core particle or the seal-coated core particle, preferably about 4 wt % to about 30 wt %, more preferably about 6 wt % to about 20 wt %, even more preferably about 7 wt % to about 15 wt %. The amount of sustained-release polymer in case of multi coating layer is about 2 wt % to about 40 wt % per the total weight of the core particle or the seal-coated core particle, preferably about 5 wt % to about 15 wt %, more preferably about 8 wt % to about 12 wt %, even more preferably about 10 wt %, but not limited thereto. The amount of sustained-release polymer in case of single coating layer is about 2 wt % to about 40 wt % per the total weight of the core particle or the seal-coated core particle, preferably about 5 wt % to about 15 wt %, more preferably about 6 wt % to about 13 wt %, but not limited thereto.

The amount of enteric polymer in both cases of multi coating layer and single coating layer is about 10 wt % to about 50 wt % per the total weight of the core particle or the seal-coated core particle, preferably about 10 wt % to about 40 wt %, more preferably about 15 wt % to about 40 wt %. The amount of enteric polymer in case of multi coating layer is about 10 wt % to about 50 wt % per the total weight of the core particle or the seal-coated core particle, preferably about 10 wt % to about 40 wt %, more preferably about 15 wt % to about 30 wt %, even more preferably about 15 wt % to about 25 wt %, but not limited thereto. The amount of enteric polymer in case of single coating layer is about 10 wt % to about 50 wt % per the total weight of the core particle or the seal-coated core particle, preferably about 15 wt % to about 40 wt %, more preferably about 20 wt % to about 40 wt %, but not limited thereto.

When the core particle is single-layer-coated with a coating liquid comprising sustained-release polymer and enteric polymer, it is desirable to use ethyl acrylate-methyl methacrylate copolymer as sustained-release polymer, and methacrylic acid copolymer LD as enteric polymer. The amount of the mixture of sustained-release polymer and enteric polymer is about 20 wt % to about 75 wt % per the total weight of the core particle or the seal-coated core particle, preferably about 25 wt % to 70 wt %, more preferably about 25 wt % to about 65 wt %. In a coating liquid comprising sustained-release polymer and enteric polymer, the weight ratio of the sustained-release polymer to the enteric polymer is about 1:1 to about 1:5, preferably about 1:2 to about 1:4, more preferably about 1:3.

The coating liquid used herein (i.e., a coating liquid comprising a sustained-release polymer, a coating liquid comprising an enteric polymer, a coating liquid comprising sustained-release polymer and enteric polymer) may comprise a surfactant, a pore former, a plasticizer, an anti-tacking agent, or the like.

The surfactant herein includes the surfactant that may be comprised in the core particle described above. Preferably, the surfactant may include polysorbate 80.

The amount of surfactant is about 1 wt % to about 25 wt % per the weight of the sustained-release polymer, preferably about 5 wt % to about 20 wt %, more preferably about 10 wt %.

The pore former includes a hydrophilic polymer, for example, hydroxypropylcellulose, hypromellose (HPMC), polyethylene glycol, poloxamer 188, polyvinylpyrrolidone, d-mannitol, methylcellulose, polyvinyl alcohol-polyethylene glycol graft copolymer, and saccharides, but not limited thereto. Preferably, it may include one or more selected from hypromellose (HPMC), hydroxypropylcellulose, polyvinylpyrrolidone, and methylcellulose. More preferably, the pore former may include one or more selected from hypromellose (HPMC), and methylcellulose.

The amount of pore former is about 0.5 wt % to about 20 wt % per the weight of the sustained-release polymer, preferably about 1 wt % to about 18 wt %, more preferably about 2.5 wt % to about 16 wt %.

The plasticizer herein includes the plasticizer that may be comprised in the core particle described above. Preferably, the plasticizer may include triethyl citrate.

The amount of plasticizer is about 5 wt % to about 40 wt % per the weight of the polymer in each coated layer, preferably about 10 wt % to about 25 wt %.

The anti-tacking agent includes, for example, talc, glyceryl monostearate, fumed silica (e.g. AEROSIL™ 200 available from Evonik Industries), precipitated silica (e.g. SIPERNAT™ PQ), and magnesium stearate. Preferably the anti-tacking agent may include talc.

The amount of anti-tacking agent is about 20 wt % to about 150 wt % per the weight of the polymer in each coated layer, preferably about 25 wt % to about 100 wt %

The pharmaceutical composition of the present invention can be prepared by optionally coating a core particle comprising Compound (I) or a salt thereof with a seal coating solution, and then spraying a coating liquid comprising at least two polymers to the particle in a known manner.

The product may be dried as needed, after the coating of a coating liquid. The drying may be done by a conventional drying method for drug formulations such as vacuum drying, fluidized bed drying, and tray drying.

The core particles can be characterized by having a distribution of particle sizes.

In the present invention, the upper limitation of the particle size of the core particles is 3 mm or less, preferably 2 mm or less, more preferably 1 mm or less, but it may be correlated with the mean particle size of the core particles. And, in the formulation of the present invention, the lower limitation of the particle size of the core particles is, but not limited to, preferably 0.3 mm or more, more preferably 0.5 mm or more.

At least a portion or all of the particles prepared by coating the core particles or seal-coated core particles with a coating liquid comprising at least two polymers have a size of about 0.2 mm to about 2 mm, preferably about 0.3 mm to about 1.5 mm, more preferably about 0.4 mm to about 1.0 mm, but should not be limited thereto.

The pharmaceutical composition of the present invention can be also characterized by dissolution profile which is evaluated through dissolution test, and it is possible to predict the actual pharmacokinetics with the dissolution profile. An example of the conditions for dissolution test is shown below, but the conditions are not necessarily limited thereto.

The dissolution test of the present pharmaceutical composition comprising a salt of Compound (I) may be carried out according to Test Method 1 (rotating basket method) in the Japanese Pharmacopoeia 18th Ed. using 900 mL of 0.1 N hydrochloric acid as a dissolution medium, at 37° C.±0.5° C. at 100 rpm for 2 hours. After 2 hours, the dissolution medium is removed off, pH 7.4 phosphate buffer at 37° C.±0.5° C. is added to the test vessel, and the dissolution test is restarted immediately. The test is done for 10 hours under conditions of 37° C.±0.5° C. and 100 rpm.

A preferred dissolution profile of the present pharmaceutical composition can be characterized by a release rate, for example, as optionally selected, less than about 10% release of Compound (1) at 2 hours after test initiation, and further as optionally selected, about 80% or more release at 4-12 hours.

Preferably, it can be characterized by a release rate, for example, as optionally selected, less than about 10% release of Compound (1) at 2 hours after test initiation, further as optionally selected, about 30% or more release at 4 hours, further as optionally selected, and about 80% or more release at 12 hours or before.

The oral solid dosage formulation comprising the present pharmaceutical composition can be used in the form of capsules or granules. To the present pharmaceutical composition, pharmaceutical formulation ingredients (additives) can be added as needed, and the mixture can be processed into a solid oral formulation comprising the present pharmaceutical composition, such as capsules and granules. Capsules as used herein refer to formulations containing the present pharmaceutical composition in capsules.

The present pharmaceutical composition can be used in combination with other sustained release particle and/or immediate release particle comprising Compound (I) or a salt thereof, and can be used as a solid oral formulation such as capsules and granules.

The oral solid dosage formulation comprises the pharmaceutical composition of the present invention, and may further comprise additional sustained release particle and/or immediate release particle which comprise Compound (I) or a salt thereof, and additives.

The sustained release particle comprising Compound (I) or a salt thereof is characterized by having an inner core and an outer layer, wherein the inner core is a “core particle comprising Compound (I) or a salt thereof” and the outer layer is a “coating-layer comprising sustained-release polymer”. And, the core particle comprising Compound (I) or a salt thereof may be seal-coated.

And, the sustained release particle can be prepared according to the composition described in the above explanation of the delayed-release (enteric) particle, for example, it can be prepared by coating a core particle with a coating liquid comprising a sustained-release polymer. The components contained in the core particle and their amounts, and the components contained in the sustained-release polymer and their amounts may be the components and their amounts described above for the delayed-release (enteric) particle.

The immediate release particle comprising Compound (I) or a salt thereof is characterized by having only an inner core, wherein the inner core is a “core particle comprising Compound (I) or a salt thereof”.

The amount of Compound (I) or a salt thereof in the immediate release particle is about 5 wt % to about 95 wt % per the total weight of the immediate release particle, preferably about 5 wt % to about 90 wt %, more preferably about 5 wt % to about 85 wt %, even more preferably about 10 wt % to about 80 wt %, the most preferably about 10 wt % to about 50 wt %.

In addition, the immediate release particle may comprise the additives for the core particle contained in the above delayed-release (enteric) particle, and may contain such amounts.

The pharmaceutical composition of the present invention is effective for diseases to be prevented and/or treated by inhibiting the reuptake of plural biogenic amines which relate as a cause of target central nervous system disorder, which includes the followings:
(i) attention deficit hyperactivity disorder (ADHD, in both children and adults) and related behavioral disorders, and forms and symptoms of alcohol abuse, drug abuse, obsessive-compulsive disorder, learning disabilities, reading problem, gambling addiction, mania, phobias, panic attacks, oppositional defiant disorder, conduct disorder, disruptive behavior disorder, academic problems in school, smoking, deviant sexual behavior, schizophrenic behavior, somatization, depression (major depressive disorder, resex; dysthymic disorder; depressive disorder not specified (NOS); major depressive disorder, single episode; depression associated with bipolar disorder, Alzheimer's disease, psychosis or Parkinson's disease; postpartum depression; and seasonal affective disorder, but not limited thereto), sleep disorders, generalized anxiety, stuttering and tics (such as Tourette's disorder);
(ii) ADHD, substance abuse, depression, anxiety disorder (including panic disorder, generalized anxiety disorder, obsessive-compulsive disorder, posttraumatic stress disorder, and social anxiety disorder, but not limited thereto), autism, traumatic brain injury, cognitive impairment, schizophrenia (especially, in cognition), obesity, chronic pain disorder, personality disorder, and mild cognitive impairment;
(iii) anxiety disorder, panic disorder, posttraumatic stress disorder, obsessive-compulsive disorder, schizophrenia and related disorder, obesity, tic disorder, intoxication, Parkinson's disease, and chronic pain;
(iv) substance abuse disorder (including alcohol-related disorder, nicotine-related disorder, amphetamine-related disorder, cannabis-related disorder, cocaine-related disorder, hallucinogen use disorder, inhalant-related disorder, and opioid-related disorder, but not limited thereto);
(v) cognitive impairment, bipolar disorder, anorexia nervosa, bulimia nervosa, cyclothymic disorder, chronic fatigue syndrome, chronic or acute stress, fibromyalgia and other somatoform disorders (such as somatization disorder, conversion disorder, pain disorder, hypochondriasis, body dysmorphic disorder, undifferentiated somatoform disorder, and somatoform NOS), incontinence (i.e., tonic incontinence, genuine stress incontinence, and mixed incontinence), inhalation disorder, mania, migraine, peripheral neuropathy;
(vi) addictive disorder (including eating disorder, impulse control disorder, alcohol-related disorder, nicotine-associated condition, amphetamine-related disorder, cannabis-related disorder, cocaine-related disorder, hallucinogen use disorder, inhalant-related disorder, and opioid-related disorder, but not limited thereto)
(vii) fragile X-associated disorder;
(viii) autism spectrum disorder (ASD), for example, ASD in a patient suffering from fragile X-associated disorder;
(ix) ADHD in a patient suffering from fragile X-associated disorder;
(x) comorbidity of ADHD and depression;
(xi) comorbidity of ADHD and substance abuse;
(xii) comorbidity of ADHD and anxiety disorder.

In another embodiment, the pharmaceutical composition of the present invention may be effective for treating any one of the following disorder.
attention deficit hyperactivity disorder (ADHD) and related behavioral disorders, and forms and symptoms of substance abuse (such as alcohol abuse and drug abuse), obsessive-compulsive behavior, learning disabilities, reading problem, gambling addiction, mania, phobias, panic attack, defiant behavior, conduct disorder, academic problems in school, smoking, deviant sexual behavior, schizophrenic behavior, somatization, depression, sleep disorder, generalized anxiety, stuttering and tics;
depression, anxiety disorder, autism, traumatic brain injury, cognitive impairment, schizophrenia (especially, in cognitive impairment), obesity, chronic pain disorder, personality disorder, and mild cognitive impairment;
panic disorder, posttraumatic stress disorder, obsessive-compulsive disorder, schizophrenia and related disorder, obesity, tic disorder, and Parkinson's disease; fragile X-associated disorder;
fragile X-associated disorder in which patients were refractory to prior treatment of fragile X-associated disorder;
attention deficit/hyperactivity disorder (ADHD) which is, for example, comorbid with anxiety and/or depression (for example, depression) in patients suffering from fragile X-associated disorder;
autism spectrum disorder (ASD).

In another embodiment, the disorder to be tried to be prevented and/or treated with the pharmaceutical composition of the present invention includes disorders described in Quick Reference to the Diagnostic Criteria from DSM-IV, American Psychiatric Association, Washington D.C., 1994. The target disorder includes attention deficit/hyperactivity disorder, predominantly inattentive type; attention deficit/hyperactivity disorder, predominantly hyperactive-impulsive type; attention deficit/hyperactivity disorder, mixed-type; attention deficit/hyperactivity disorder not specified (NOS); conduct disorder; oppositional defiant disorder; and disruptive behavior disorder not specified (NOS), but not limited thereto.

The depressive disorder in the present invention includes major depressive disorder, recurrent depression, dysthymic disorder, depressive disorder not specified (NOS), major depressive disorder, and single episode, but not limited thereto.

The addictive disorder in the present invention includes eating disorder, impulse control disorder, alcohol-related disorder, nicotine-related disorder, amphetamine-related disorder, cannabis-related disorder, cocaine-related disorder, hallucinogen use disorder, inhalant-related disorder, and opioid-related disorder, but not limited thereto.

The dose of the present pharmaceutical composition should not be particularly limited, but it varies depending on the subject of administration, administration route, and symptoms. For example, when orally administered to an adult patient of normal body weight (about 40 to 80 kg in weight), the amount of the active ingredient Compound (I) or a salt thereof is about 0.05-30 mg/kg body weight per day, preferably may be administered at a dose range of about 0.2-20 mg/kg body weight. For example, in the case of oral administration to pediatric patients (for example, body weight of less than 40 kg), the amount of the active ingredient Compound (I) or a salt thereof is about 0.01-20 mg/kg body weight, preferably may be administered at a dose range of about 0.2-10 mg/kg body weight per day.

The administration frequency is once, twice or more daily.

The specific regimen, i.e., dose, timing, and repetition, depends on the particular individual and the medical history of the individual.

### EXAMPLES

### Example 1

3,200 g of Compound (I) hydrochloride (a product micronized with a screen mill) and 800 g of microcrystalline cellulose (CEOLUS™ PH-301: AsahiKASEI) were put into a stirring granulator (VG-25: POWREX), and then 1,020 g of purified water was added to the granulator while mixing with stirring. The obtained mixture was extrusion-granulated with an extrusion granulator (TDG-80: DALTON) equipped with dome dies whose hole diameter is 0.6 mm, and then made to be spherical particles with a spheronizer (QJ-700: Fuji Paudal). The obtained particle was dried with a fluid bed granulator (Flo-5M: FREUND), and screened with two sieves whose sieve opening sizes are 0.71 mm and 0.5 mm to give a core particle whose diameter is 0.5 to 0.71 mm.

### Example 2

15 g of hypromellose (Methocel™ E5 Premium LV: Du Pont) was dissolved in 285 g of purified water to prepare a coating liquid. 400 g of the core particle prepared in Example 1 was put into a fluid bed granulator (MP-01: POWREX), and then coated with the coating liquid prepared above until the coating ingredient was 3% of the core particle weight. The coated particle was dried in the fluid bed granulator to give a seal-coated particle.

### Example 3

7.5 g of hypromellose (Methocel™ E5 Premium LV: Du Pont) and 5 g of polysorbate 80 (Tween™ 80: Merck) were dissolved in 445.8 g of purified water. 50 g of talc (Luzenac Pharma™ M: Imerys) was dispersed in the above-prepared solution and then 166.7 g of ethyl acrylate-methyl methacrylate copolymer dispersion (Eudragit™ NM30D: Evonik) was added and stirred to prepare a coating dispersing liquid. 400 g of the seal-coated particle prepared in Example 2 was put into a fluid bed granulator (MP-01: POWREX), and then coated with the coating dispersing liquid prepared above until the coating ingredient was 22.5% of the seal-coated particle weight. The coated particle was taken out from the fluid bed granulator, and dried at 50° C. for 24 hours in a tray oven to give a sustained-release coated particle.

### Example 4

333.3 g of methacrylic acid copolymer LD (Eudragit™ L30D-55: Evonik), 10 g of triethyl citrate (Triethyl citrate: Merck), and 50 g of talc (Luzenac Pharma™ M: Imerys) were dispersed in 406.7 g of purified water to prepare a coating liquid. 400 g of the sustained-release coated particle prepared in Example 3 was put into a fluid bed granulator (MP-01: POWREX), and then coated with the coating liquid prepared above until the coating ingredient was 24% or 32% of the sustained-release coated particle weight to give two types of delayed-release (enteric) particles (Example 4-1 is a particle whose coating rate is 24%, and Example 4-2 is a particle whose coating rate is 32%).

TABLE 1
Compositions of Examples 4-1 and 4-2 (mg/unit)

| | | Example | Example |
|---|---|---|---|
| Component | Ingredient | 4-1 | 4-2 |
| Core | Compound (I) hydrochloride | 100.00 | 100.00 |
| particle | microcrystalline cellulose | 25.00 | 25.00 |
| Seal coat | hypromellose | 3.75 | 3.75 |
| Sustained- | ethyl acrylate-methyl | 12.88 | 12.88 |
| release | methacrylate copolymer | | |
| coat | talc | 12.88 | 12.88 |
| | hypromellose | 1.93 | 1.93 |
| | polysorbate 80 | 1.29 | 1.29 |
| Enteric | methacrylic acid copolymer LD | 23.66 | 31.54 |
| coat | triethyl citrate | 2.37 | 3.15 |
| | talc | 11.83 | 15.77 |

### Example 5

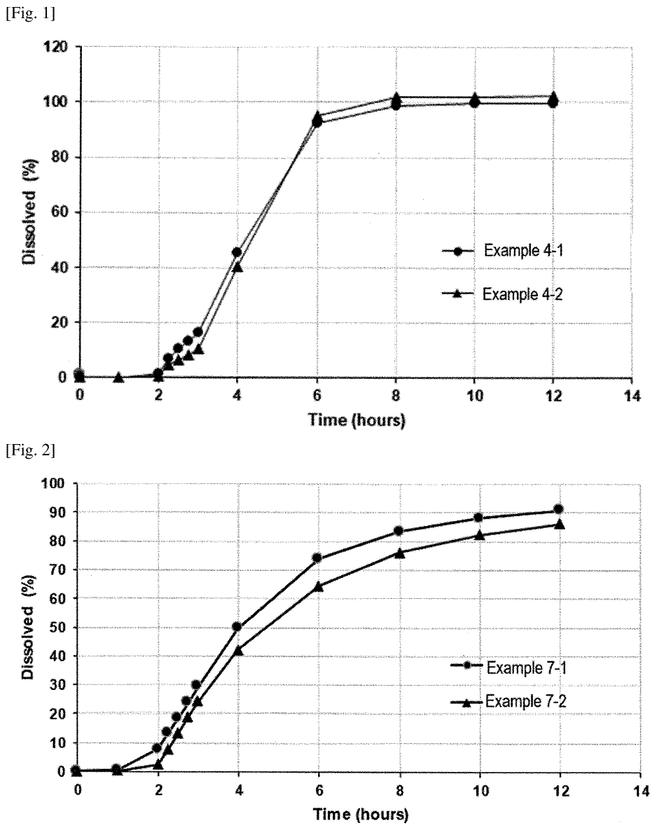

The release rate of the particle prepared in Example 4 was evaluated by dissolution test including Acid stage and Buffer stage. The dissolution test was carried out according to Test Method 1 (rotating basket method) in the Japanese Pharmacopoeia 18th Ed. The test was performed for 2 hours at Acid stage, the dissolution medium was removed off after completing the Acid stage test, the dissolution medium was switched to Buffer stage, and the dissolution test of the same sample was continued at Buffer stage. The Buffer stage test was performed for 10 hours. As the dissolution medium, 900 mL of 0.1 N hydrochloric acid was used at Acid stage, and 900 mL of pH 7.4 phosphate buffer was used at Buffer stage, wherein the temperature of the dissolution medium was 37° C., and the rotation speed was 100 rpm. The sampling was done over time. The amount of Compound (I) (centanafadine free base) in the sampled solution was measured with a UV detector (wave length of absorbance measurement: 276 nm and 350 nm). The dissolution rate (%) was defined as the rate of the weight of dissolved Compound (I) per the total weight of Compound (I) contained initially in the formulation. The dissolution rate at Buffer stage was analyzed by accumulating Acid stage and Buffer stage. The result is shown in FIG. 1.

### Example 6

6.2 g of methylcellulose (Metolose™ SM-4: Shin-Etsu Chemical) and 10.3 g of triethyl citrate (Triethyl citrate: Merck) were dissolved in 231.0 g of purified water. 137.5 g of an aqueous dispersion of ethylcellulose (Aquacoat™ ECD-30: Du Pont) was dispersed in the above-prepared solution to prepare a coating liquid. 400 g of the seal-coated particle prepared in Example 2 was put into a fluid bed granulator (MP-01: POWREX), and then coated with the coating liquid prepared above until the coating ingredient was 14% of the seal-coated particle weight. The coated particle was taken out from the fluid bed granulator, and dried at 60° C. for 18 hours in a shelf dryer to give a sustained-release coated particle.

### Example 7

666.7 g of methacrylic acid copolymer LD (Eudragit™ L30D-55: Evonik), 20 g of triethyl citrate (Triethyl citrate: Merck), and 100 g of talc (Luzenac Pharma™ M: Imerys) were dispersed in 813.3 g of purified water to prepare a coating liquid. The sustained-release coated particle prepared in Example 6 was put into a fluid bed granulator (MP-01: POWREX), and then coated with the coating liquid prepared above until the coating ingredient was 24% or 31% of the sustained-release coated particle weight to give two types of delayed-release (enteric) particles (Example 7-1 is a particle whose coating rate is 24%, and Example 7-2 is a particle whose coating rate is 31%).

TABLE 2
Compositions of Examples 7-1 and 7-2 (mg/unit)

| | | Example | Example |
|---|---|---|---|
| Component | Ingredient | 7-1 | 7-2 |
| Core | Compound (I) hydrochloride | 100.00 | 100.00 |
| particle | microcrystalline cellulose | 25.00 | 25.00 |
| Seal coat | hypromellose | 3.75 | 3.75 |
| Sustained- | ethylcellulose | 12.88 | 12.88 |
| release | methylcellulose | 1.93 | 1.93 |
| coat | triethyl citrate | 3.22 | 3.22 |
| Enteric | methacrylic acid copolymer LD | 22.02 | 28.62 |
| coat | triethyl citrate | 2.2 | 2.86 |
| | talc | 11.01 | 14.31 |

### Example 8

Each particle in Table 2 was evaluated in a similar means to the dissolution test described in Example 5. The result is shown in FIG. 2.

### Example 9

10 g of polysorbate 80 (Tween™ 80: Merck) was dissolved in 1,106.7 g of purified water. 1,000 g of methacrylic acid copolymer LD (Eudragit™ L30D-55: Evonik), 333.3 g of ethyl acrylate-methyl methacrylate copolymer dispersion (Eudragit™ NM30D: Evonik), and 100 g of talc (Luzenac Pharma™ M: Imerys) were dispersed in the above-prepared solution to prepare a coating dispersing liquid. 400 g of the seal-coated particle prepared in Example 2 was put into a fluid bed granulator (MP-01: POWREX), and then coated with the coating dispersing liquid prepared above until the coating ingredient was 38.3% or 63.8% of the seal-coated particle weight. The coated particle was taken out from the fluid bed granulator, and dried at 50° C. for 24 hours in a shelf dryer to give two types of delayed-release (enteric) particles (Example 9-1 is a particle whose coating rate is 38.3%, and Example 9-2 is a particle whose coating rate is 63.8%).

TABLE 3
Compositions of Examples 9-1 and 9-2 (mg/unit)

| | | Example | Example |
|---|---|---|---|
| Component | Ingredient | 9-1 | 9-2 |
| Core | Compound (I) hydrochloride | 100.00 | 100.00 |
| particle | microcrystalline cellulose | 25.00 | 25.00 |
| Seal coat | hypromellose | 3.75 | 3.75 |
| Sustained- | methacrylic acid copolymer LD | 28.97 | 48.28 |
| release/ | ethyl acrylate-methyl | 9.66 | 16.09 |
| enteric | methacrylate copolymer | | |
| coat | polysorbate 80 | 0.97 | 1.61 |
| | talc | 9.66 | 16.09 |

### Example 10

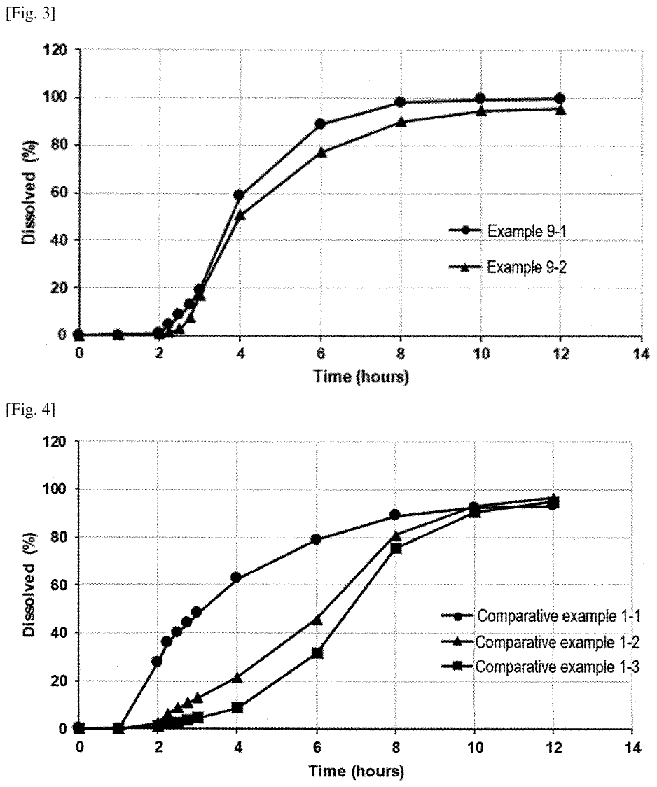

Each particle in Table 3 was evaluated in a similar means to the dissolution test described in Example 5. The result is shown in FIG. 3.

### Comparative Example 1

30 g of polysorbate 80 (Tween™ 80: Merck) was dissolved in 1,986.7 g of purified water. 333.3 g of methacrylic acid copolymer LD (Eudragit™ L30D-55: Evonik), 1,000 g of ethyl acrylate-methyl methacrylate copolymer dispersion (Eudragit™ NM30D: Evonik), and 300 g of talc (Luzenac Pharma™ M: Imerys) were dispersed in the above-prepared solution to prepare a coating dispersing liquid. 400 g of the seal-coated particle prepared in Example 2 was put into a fluid bed granulator (MP-01: POWREX), and then coated with the coating dispersing liquid prepared above until the coating ingredient was 36.5%, 45.63%, or 54.8% of the seal-coated particle weight. The coated particle was taken out from the fluid bed granulator, and dried at 50° C. for 24 hours in a shelf dryer to give three types of particles (Comparative example 1-1 is a particle whose coating rate is 36.5%, Comparative example 1-2 is a particle whose coating rate is 45.63%, and Comparative example 1-1 is a particle whose coating rate is 54.8%).

TABLE 4
Compositions of Comparative examples 1-1 to 1-3 (mg/unit)

| | | Compar- | Compar- | Compar- |
|---|---|---|---|---|
| | | ative | ative | ative |
| | | exam- | exam- | exam- |
| Component | Ingredient | ple 1-1 | ple 1-2 | ple 1-3 |
| Core | Compound (I) | 100.00 | 100.00 | 100.00 |
| particle | hydrochloride | | | |
| | microcrystalline | 25.00 | 25.00 | 25.00 |
| | cellulose | | | |
| Seal coat | hypromellose | 3.75 | 3.75 | 3.75 |
| Sustained- | methacrylic acid | 6.44 | 8.05 | 9.66 |
| release/ | copolymer LD | | | |
| enteric | ethyl acrylate- | 19.31 | 24.14 | 28.97 |
| coat | methyl methacrylate | | | |
| | copolymer | | | |
| | polysorbate 80 | 1.93 | 2.41 | 2.90 |
| | talc | 19.31 | 24.14 | 28.97 |

### Comparative Example 2

Each particle in Table 4 was evaluated in a similar means to the dissolution test described in Example 5. The result is shown in FIG. 4.

1. A pharmaceutical composition comprising a core particle with a coating layer, wherein the core particle comprises (1R,5S)-1-(naphthalen-2-yl)-3-azabicyclo[3.1.0]hexane or a salt thereof as an active ingredient, and wherein the coating layer comprises at least two types of polymers.

2. The pharmaceutical composition of claim 1, wherein the two types of polymers are an enteric polymer and a sustained-release polymer.

3. The pharmaceutical composition of claim 2, wherein the enteric polymer is methacrylic acid copolymer LD.

4. The pharmaceutical composition of claim 2, wherein the sustained-release polymer comprises one or more polymer(s) selected from alkylcellulose, acrylic acid polymer, acrylic acid copolymer, vinyl acetate polymer and cellulose ether.

5. The pharmaceutical composition of claim 4, wherein the sustained-release polymer comprises one or more polymer(s) selected from alkylcellulose and acrylic acid copolymer.

6. The pharmaceutical composition of claim 5, wherein the alkylcellulose is ethylcellulose, or wherein the acrylic acid copolymer is ethyl acrylate-methyl methacrylate copolymer.

7. (canceled)

8. The pharmaceutical composition of claim 2, wherein the enteric polymer is methacrylic acid copolymer LD, and wherein the sustained-release polymer is ethylcellulose or wherein the enteric polymer is methacrylic acid copolymer LD, and wherein the sustained-release polymer is ethyl acrylate-methyl methacrylate copolymer.

9. (canceled)

10. (canceled)

11. The pharmaceutical composition of claim 1, wherein the coating layer is composed of two coating layers: a coating layer comprising enteric polymer and a coating layer comprising sustained-release polymer, wherein the coating layer of enteric polymer covers the coating layer of sustained-release polymer.

12. The pharmaceutical composition of claim 11, wherein the amount of sustained-release polymer in case of multi coating layer is about 2 wt % to about 40 wt %, or about 5 wt % to about 15 wt %, or about 8 wt % to about 12 wt %, or about 10 wt % per the total weight of the core particle or the seal-coated core particle.

13. The pharmaceutical composition of claim 11, wherein the amount of enteric polymer in case of multi coating layer is about 10 wt % to about 50 wt %, or about 10 wt % to about 40 wt %, or about 15 wt % to about 30 wt %, or about 15 wt % to about 25 wt % per the total weight of the core particle or the seal-coated core particle.

14. (canceled)

15. The pharmaceutical composition of claim 1, wherein the coating layer is a single layer, and the single coating layer comprises sustained-release polymer and enteric polymer.

16. The pharmaceutical composition of claim 15, wherein the amount of sustained-release polymer in case of single coating layer is about 2 wt % to about 40 wt %, or about 5 wt % to about 15 wt %, or about 6 wt % to about 13 wt % per the total weight of the core particle or the seal-coated core particle.

17. The pharmaceutical composition of claim 15, wherein the amount of enteric polymer in case of single coating layer is about 10 wt % to about 50 wt %, or about 15 wt % to about 40 wt %, or about 20 wt % to about 40 wt % per the total weight of the core particle or the seal-coated core particle.

18. The pharmaceutical composition of claim 15, wherein the weight ratio of the sustained-release polymer to the enteric polymer in the coating layer is 1:1 to 1:5.

19. (canceled)

20. The pharmaceutical composition of claim 1, wherein the core particle is seal-coated.

21. The pharmaceutical composition of claim 1, wherein the core particle with the coating layer is a delayed-release (enteric) particle.

22. The pharmaceutical composition of claim 1, wherein the active ingredient is (1R,5S)-1-(naphthalen-2-yl)-3-azabicyclo[3.1.0]hexane hydrochloride.

23. The pharmaceutical composition of claim 1, which meets the followings:
a) less than 10% of the active ingredient is released at 2 hours, and
b) 80% or more of the active ingredient is released at 4 to 12 hours,
when the pharmaceutical composition is tested according to Apparatus for Basket Method in the Japanese Pharmacopoeia dissolution test at 37° C.±0.5° C. at 100 rpm, first in 900 mL of 0.1 N HCl solution for 2 hours, and then in 900 mL of pH 7.4 buffer solution for 10 hours; or wherein
a) less than 10% of the active ingredient is released at 2 hours,
b) 30% to 70% of the active ingredient is released at 4 hours, and
c) 80% or more of the active ingredient is released at 12 hours,
when the pharmaceutical composition is tested according to Apparatus for Basket Method in the Japanese Pharmacopoeia dissolution test at 37° C.±0.5° C. at 100 rpm, first in 900 mL of 0.1 N HCl solution for 2 hours, and then in 900 mL of pH 7.4 buffer solution for 10 hours.

24. (canceled)

25. (canceled)

26. (canceled)

27. (canceled)

28. An oral solid dosage formulation comprising the pharmaceutical composition of claim 1, optionally wherein the oral solid dosage formulation is granules or capsules.

29. (canceled)

30. The oral solid dosage formulation of claim 28, further comprising a sustained release particle comprising (1R,5S)-1-(naphthalen-2-yl)-3-azabicyclo[3.1.0]hexane or a salt thereof and/or an immediate release particle comprising (1R,5S)-1-(naphthalen-2-yl)-3-azabicyclo[3.1.0]hexane or a salt thereof.

31. A method of in preventing and/or treating central nervous system (CNS) disorder comprising administering the pharmaceutical composition of claim 1 to a subject in need thereof.

32. The method of claim 31, wherein the wherein the central nervous system (CNS) disorder is at least one CNS disease selected from the group consisting of schizophrenia; treatment-resistant, intractable, or chronic schizophrenia; emotional disorder; psychotic disorder; mood disorder; bipolar disorder; mania; depression; endogenous depression; major depression; melancholia and treatment-resistant depression; dysthymic disorder; cyclothymic disorder; anxiety disorder; somatoform disorder; factitious disorder; dissociative disorder; sexual disorder; eating disorder; sleep disorder; adjustment disorder; substance-related disorder; anhedonia; delirium; cognitive impairment; Alzheimer's disease; Parkinson's disease; cognitive impairment associated with other neurodegenerative diseases; BPSD associated with dementia; cognitive impairment in schizophrenia; cognitive impairment due to treatment-resistant, intractable, or chronic schizophrenia; vomiting; motion sickness; obesity; migraine; pain; mental retardation; autism; Tourette's disorder; tic disorder; attention deficit hyperactivity disorder; conduct disorder; borderline personality disorder; nicotine-related disorder; bulimia; and Down syndrome.
